(19) United States
(12) Patent Application Publication
(10) Pub. No.: US 2012/0289890 A1
(43) Pub. Date: Nov. 15, 2012

(54) OXYGEN DELIVERY DEVICE

(76) Inventors: Srinivasan Sarangapani, Walpole, MA (US); Lawrence J. Cali, East Falmouth, MA (US); Daniel A. Scherson, Beachwood, OH (US); Melvyn I. Burk, Beachwood, OH (US)

(21) Appl. No.: 13/457,564

(22) Filed: Apr. 27, 2012

Related U.S. Application Data
(60) Provisional application No. 61/484,686, filed on May 11, 2011.

(51) Int. Cl. A61M 35/00

(52) U.S. Cl. 604/24; 604/23; 604/26

(57) ABSTRACT

A portable topical oxygen therapy system includes a miniature gas cylinder containing compressed oxygen, a miniature regulator or having an inlet and an outlet and a wound dressing having an interior configured to cover or enclose an exterior portion of a patient's body. The inlet of the regulator is connected to the gas cylinder. The interior of the wound dressing is connected to the outlet of the regulator. The portable topical oxygen therapy system is sized and configured to be wearable by the patient without interfering with normal ambulation.

### CROSS-REFERENCE TO RELATED APPLICATIONS

This application claims the priority benefit of U.S. Application Ser. No. 61/484,686, filed May 11, 2011. The disclosure of U.S. Application Ser. No. 61/484,686 is incorporated herein by reference in its entirety.

### BACKGROUND

The present disclosure relates to oxygen delivery devices, more particularly to portable oxygen delivery devices.

Wound healing is a complex biological process that requires the successful mobilization and integration of cells to repopulate the wound. Optimal metabolic function of these cells is critical for the success of this process which implies that oxygen must be available. Chronic wound ischemia is a pathological condition that inhibits normal wound healing. Oxygen plays an essential role in energy metabolism and is also important for polymorphonuclear cell function, neovascularization, fibroblast proliferation, and collagen deposition. Each of these functions is critical in wound repair.

Needed oxygen can be delivered to the wound using three different modalities: systemic, topical, and transdermal routes. Systemic oxygen therapy is administered using hyperbaric chambers. Topical oxygen therapy uses collapsible or rigid plastic chambers to provide oxygen for the extremities. In the transdermal route of administration, oxygen is delivered across the skin.

A fuel cell-based approach may be used to deliver oxygen for continuous oxygen therapy as disclosed for example in U.S. Pat. Nos. 5,578,022, 7,429,252, and 8,088,113. U.S. Pat. No. 7,263,814 teaches a balloon concept for delivering oxygen to a wound site. These disclosures of these patents are incorporated by reference herein in their entireties.

It would be desirable to develop new devices, systems, and methods for providing oxygen to wounds.

### BRIEF DESCRIPTION

The present disclosure relates to oxygen delivery devices, more particularly to portable oxygen delivery devices.

Disclosed in embodiments is a portable topical oxygen therapy system. The system includes a miniature gas cylinder containing compressed oxygen, a miniature pressure regulator having an inlet and an outlet, and a wound dressing having an interior configured to cover or enclose an exterior portion of a patient's body. The inlet of the pressure regulator is connected to the gas cylinder. The interior of the wound dressing is connected to the outlet of the pressure regulator. The portable topical oxygen therapy system is sized and configured to be wearable by the patient without interfering with normal ambulation.

The gas cylinder may have an empty weight of about 50 to about 70 grams. The gas cylinder may have an internal volume of about 10 cm³. In some embodiments, the gas cylinder is pressure rated up to 1000 psig.

The gas cylinder may further contain one or more components selected from the group consisting of nitric oxide (NO), chlorine dioxide (ClO₂), ozone (O₃), and nitrogen (N₂).

In some embodiments, the regulator has a rated output pressure of 0 to about 5 psig. The system may further include a shut-off valve between the gas cylinder and the regulator. In some embodiments, the system further includes a cannula connected to the wound dressing. The cannula may be connected to a needle valve.

The total weight of the system may be about 250 grams to about 350 grams. In some embodiments, the total weight of the system is less than or equal to 300 grams.

The regulator may be configured to deliver oxygen at a rate of about 0.5 to about 20.0 mL/hr, including about 0.5 to about 5.0 mL/hr. In some embodiments, the regulator is configured to deliver oxygen at a rate of about 3.0 mL/hr.

Also disclosed in embodiments is a portable oxygen delivery device including a miniature gas cylinder, an electrochemical oxygen regulator, a cannula, and a pressure regulator. The cylinder includes a cylinder outlet which is connected to the pressure regulator. An outlet of the electrochemical oxygen regulator is connected to the cannula.

Further disclosed in embodiments is a portable oxygen delivery device including a miniature gas cylinder, an electrochemical oxygen regulator, and a cannula. The cylinder includes a cylinder outlet which is connected to an inlet of the electrochemical oxygen regulator. An outlet of the electrochemical oxygen regulator is connected to the cannula.

The device may include one or more temperature sensors, one or more pressure sensors, and/or one or more flow sensors. In some embodiments, the device further includes a bandage with a diffuser.

These and other non-limiting aspects and/or subjects of the disclosure are more particularly described below.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 illustrates a schematic view of a first embodiment of a portable oxygen delivery device according to the present disclosure.

FIG. 2 illustrates a schematic view of a second embodiment of a portable oxygen delivery device according to the present disclosure.

### DETAILED DESCRIPTION

A more complete understanding of the processes and apparatuses disclosed herein can be obtained by reference to the accompanying drawings. These figures are merely schematic representations based on convenience and the ease of demonstrating the existing art and/or the present development, and are, therefore, not intended to indicate relative size and dimensions of the assemblies or components thereof.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

The modifier “about” used in connection with a quantity is inclusive of the stated value and has the meaning dictated by the context (for example, it includes at least the degree of error associated with the measurement of the particular quantity). When used with a specific value, it should also be considered as disclosing that value. For example, the term “about 2” also discloses the value “2” and the range “from about 2 to about 4” also discloses the range “from 2 to 4.”

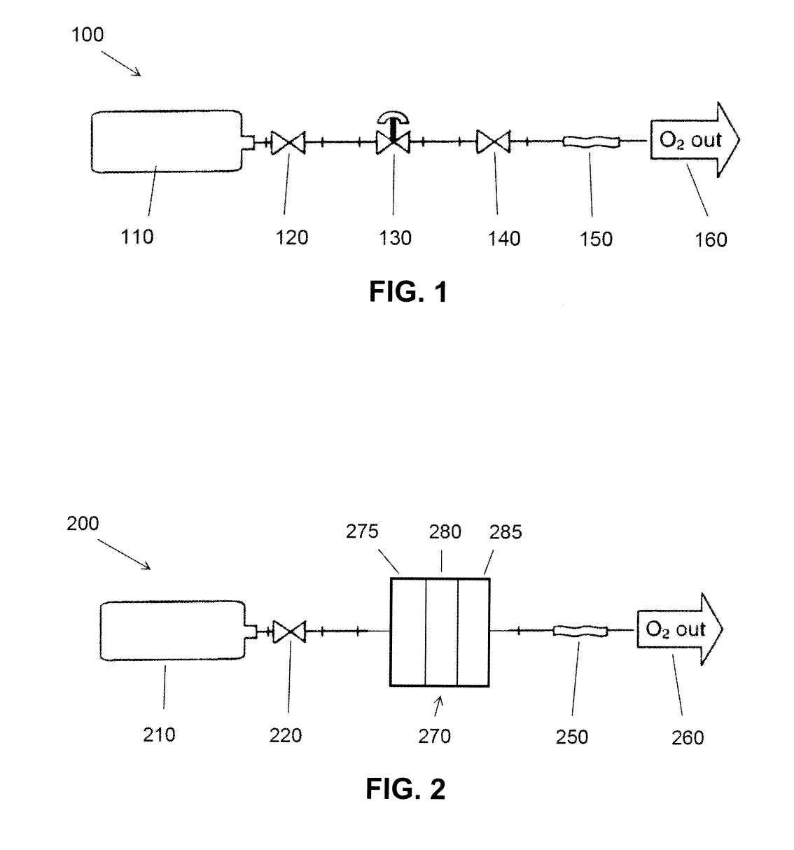

The present disclosure relates to oxygen delivery devices, more particularly to portable oxygen delivery devices. FIG. 1 illustrates a first exemplary system of the present disclosure. The system 100 includes a gas cylinder 110, a pressure regulator 130, and a cannula 150. A shut-off valve 120 is located between the gas cylinder 110 and the pressure regulator 130. To fill the gas cylinder 110 with oxygen, the shut-off valve 120 is closed and the pressure regulator 130 is disconnected. The gas cylinder 110 is then connected to an oxygen source. In some embodiments, the oxygen source is an oxygen bottle comprising USP grade oxygen with a supply pressure of 100 psig. The shut-off valve 120 is then opened to fill the gas cylinder 110 with oxygen. Then, the shut-off valve 120 is closed again and the pressure regulator 130 is reconnected. In some embodiments, disconnection of the pressure regulator to fill the gas cylinder may be avoided. For example, the device may include a fitting that can be connected to a gas supply, e.g. a larger tank or a reservoir.

The cylinders may be prefilled and delivered. In some embodiments, the cylinders are delivered to patients on a regular schedule.

The device may include a needle valve 140 to provide an adjustment to the gas flow rate. Output flow may be measured with a eudiometer (not shown) or a digital flow meter capable of reading such low flow rates. The needle valve 140 can be adjusted based on the measured flow rate. Once the flow rate is set, the needle valve 140 and pressure regulator 130 may not require any additional adjustments to maintain the flow rate. The cannula 150 guides the oxygen to an outlet flow 160. The outlet flow 160 may be directed to a wound to be treated. In some embodiments, the oxygen is directed to an area between the wound and a wound dressing. The wound dressing may have an interior configured to cover or enclose an exterior portion of a patient's body. The system is sized and configured to be wearable by the patient without interfering with normal ambulation.

The gas cylinder may be an off-the-shelf item manufactured by Swagelok part number SS-4CS-TW-10). The gas cylinder may have an internal volume of about 10 cm³ and be pressure rated to 1000 psig. The gas cylinder may be made of stainless steel, e g. 316 stainless steel. The gas cylinder may weigh about 62 grams when empty. In some embodiments, the gas cylinder includes a ⅜ of an inch diameter tube outlet suitable for compression fittings. The gas cylinder may have a diameter of about 1 inch and a length of about 2.19 inches.

The pressure regulator may be an off-the-shelf item manufactured by Beswick Engineering (part number PRD2-1N1-0-6VK). The pressure regulator may have a rated output pressure in the range of from 0 to about 5 psig, including about 0.5 psig. In some embodiments the input pressure may be as high as about 500 psig. The pressure regulator may be made of stainless steel, e.g. 316 stainless steel. The pressure regulator may weigh about 40 to about 60 grams, including about 50 grams. In some embodiments, the pressure regulator includes seals and diaphragms made from a fluoroelastomer such as VITON® (commercially available from DuPont).

Connections between components may be made using compression fittings such as those available from Swagelok. The total weight of the system may be less than or equal to about 300 grams.

In some embodiments, the cylinder and/or the regulator are made of titanium or a corrosion resistant alloy. The cylinder and/or the regulator may include a coating or plastic lining, particularly a corrosion resistant coating or plastic lining.

FIG. 2 illustrates a second exemplary system of the present disclosure. The system 200 includes a gas cylinder 210, a shut-off valve 220, and a cannula 250. The depicted embodiment 200 differs from that of FIG. 1 because the pressure regulator 130 and needle valve 140 of FIG. 1 have been replaced by an electrochemical oxygen regulator 270. The electrochemical oxygen regulator 270 comprises a cathode 275 and membrane 280, and an anode 285. The cathode 275 may be a gas permeable cathode. Oxygen from the cylinder 210 may be dead-ended into the cathode compartment. In such an embodiment, the delivery of oxygen from the cylinder will be dictated by the demand of flow required/set at the output side. Oxygen brought into contact with the cathode 275 may be reduced to neutral species such as water or hydrogen peroxide or ions, e.g. superoxide ions. These species may diffuse through the membrane 280 and be oxidized t the anode 285 to produce diatomic oxygen.

The electrochemical process may be driven by an internal or external power source. The regulation rate of the electrochemical regulator 270 may be varied by adjusting the current provided thereto and/or reversing the polarity. The current passing through the electrochemical regulator provides direct indication of oxygen flow rate. The electrochemical regulator 270 may further provide feedback on the oxygen flow rate by monitoring cell voltage as it increase when oxygen flow is below a set point. Current may also be varied in response to either a pressure or a flow sensor.

In some embodiments, the device includes a mechanical pressure regulator and an electrochemical regulator. In these embodiments, the needle valve 140 of the system of FIG. 1 is replaced with the electrochemical regulator.

The use of an electrochemical regulator permits precise regulation of the oxygen flow rate and may reduce the size and/or weight of the device.

The devices of the present disclosure optionally include one or more sensors selected from flow sensors, pressure sensors, and temperature sensors. The sensors may be located as part of or between any of the components described above.

The wound dressing may include a diffuser configured to allow ambient air in or to permit gas flow out dependent on pressure between the wound dressing and the patient. Nitric oxide (NO), ozone (O₃), chlorine dioxide (ClO₂), nitrogen (N₂), air or other gases may be mixed with the relatively pure oxygen in the cylinder or at any point along the flow path in some embodiments. Nitric oxide encourages wound healing by a biochemical mechanism. Ozone and chlorine dioxide kill bacteria and fungus. The concentrations of nitric oxide, ozone, and/or chlorine dioxide may be at the ppm level. Diatomic nitrogen may be included in up to a significant percentage in order to reduce the oxygen concentration or pure diatomic nitrogen may be included to ensure hypoxic conditions.

In some embodiments, the electrochemical regulator is not used when the cylinder contains a gas mixture including components other than oxygen. The electrochemical regulator may filter out these “impurities”. A mechanical pressure regulator may be used instead.

Aspects of the present disclosure may be further understood by referring to the following examples. The examples are illustrative, and are not intended to be limiting embodiments thereof.

### EXAMPLES

An oxygen delivery device including a gas cylinder from Swagelok (part number SS-4CS-TW-10), a shut-off valve from Swagelok (part number SS-41S2), a pressure regulator from Beswick Engineering (part number PRD2-1N1-0-6VK), a needle valve (sample port adapter assembly from Oxigraf, Mountainview, Calif.), and a cannula from Disetronic (part number PE-110) was assembled.

The gas cylinder had a 10 cm³ internal volume and was pressure rated to 1000 psig. The cylinder was constructed of 316 stainless steel and weighed 62 grams empty. The cylinder included a ⅜ of an inch diameter tube outlet suitable for compression fitting and had a length of about 2.19 inches and a diameter of about 1 inch.

The pressure regulator had a rated output pressure of from 0 to about 5 psig and could regulate at least down to 0.5 psig. The inlet pressure could be as high as 500 psig. The regulator included a body constructed of 316 stainless steel. The regulator also included seals and a diaphragm made of the fluoroelastomer sold under the trade name VITON®. The regulator weighed about 50 grams.

Connections were made using Swagelok compression fittings. The total weight of the device was about 300 grams.

To fill the gas cylinder with oxygen, the shut-off valve was closed, the pressure regulator was disconnected, and the cylinder was connected to an oxygen bottle (USP grade oxygen) with a supply pressure of 100 psig. The shut-off valve was opened to allow the gas cylinder to be filled with oxygen. When the cylinder was filled, the shut-off valve was closed and the gas regulator was reconnected.

The gas regulator can be set to maintain an output pressure of about 5 psig. At 100 psig cylinder pressure and 5 psig regulator output pressure, the device would expected to run for approximately 20 hours at a flow rate of 3 mL/hr. This pressure was selected so that the device output rate would be relatively insensitive to pressure drop changes downstream of the needle valve. For example, such changes could occur if the device was used for wound healing and exudates built up at the end of the cannula.

In an actual test, the device was pressurized to 100 psig with oxygen and operated over a period of approximately 23 hours. The regulator was set to deliver it maximum pressure (about 9 psig). For the first 17 hours, the flow rate remained at about 2.7 mL/hr. The flow rate slowly decreased after this point and the oxygen was exhausted approximately 23 hours after the start of the operation. Without wishing to be bound by theory, this behavior could be explained by the cylinder pressure approaching the set limit pressure of the regulator.

In other applications, the cylinder pressure could be set to a higher pressure, e.g. from about 500 to about 700 psi, so that there would be enough residual pressure towards the end of the operating life to maintain a relatively constant flow. The pressure at the cannula outlet during the operating period was held constant at about 10 inches of water by submerging the cannula under water in a measuring cylinder.

The exemplary device described above demonstrated the viability of the concept of using a small gas cylinder and a regulator to provide a portable oxygen supply. The test results obtained show that the device is capable of providing low oxygen flows continuously for extended periods. It is expected that using higher cylinder pressures would allow the device to be operated continuously for longer periods, e.g. up to 7 days in some embodiments, on a single oxygen charge.

The present disclosure has been described with reference to exemplary embodiments. Obviously, modifications and alterations will occur to others upon reading and understanding the preceding detailed description. It is intended that the present disclosure be construed as including all such modifications and alterations insofar as they come within the scope of the appended claims or the equivalents thereof.

1. A portable topical oxygen therapy system, comprising:
a miniature gas cylinder containing compressed oxygen;
a miniature pressure regulator having an inlet and an outlet, the inlet of the pressure regulator connected to the gas cylinder; and
a wound dressing having an interior configured to cover or enclose an exterior portion of a patient's body, the interior of the wound dressing being connected to the outlet of the pressure regulator;
wherein the portable topical oxygen therapy system is sized and configured to be wearable by the patient without interfering with normal ambulation.

2. The system of claim 1, wherein the gas cylinder weighs about 50 grams to about 70 grams when empty.

3. The system of claim 1, wherein the gas cylinder has an internal volume of about 10 cm³.

4. The system of claim 1, wherein the gas cylinder is pressure rated up to 1000 psig.

5. The system of claim 1, wherein the regulator has a rated output pressure of from 0 to about 5 psig.

6. The system of claim 1, wherein the gas cylinder further contains one or more components selected from the group consisting of nitric oxide (NO), chlorine dioxide (ClO₂), ozone (O₃), and nitrogen (N₂).

7. The system of claim 1, further comprising:
a shut-off valve between the gas cylinder and the regulator.

8. The system of claim 1, further comprising:
a cannula connected to the wound dressing.

9. The system of claim 8, further comprising:
a needle valve between the regulator and the cannula.

10. The system of claim 1, wherein the total weight of the system is about 250 grams to about 350 grams.

11. The system of claim 1, wherein the total weight of the system is less than or equal to about 300 grams.

12. The system of claim 1, wherein the regulator is configured to deliver oxygen at a rate of about 0.5 to about 20.0 mL/hr.

13. The system of claim 1, wherein the regulator is configured to deliver oxygen at a rate of about 0.5 to about 5.0 mL/hr.

14. The system of claim 1, wherein the regulator is configured to deliver oxygen at a rate of about 3.0 mL/hr.

15. A portable oxygen delivery device comprising:
a miniature gas cylinder comprising a cylinder outlet;
an electrochemical oxygen regulator comprising a regulator inlet and a regulator outlet;
a cannula connected to the regulator outlet; and
a pressure regulator between the gas cylinder and the oxygen regulator.

16. A portable oxygen delivery device comprising:
a miniature gas cylinder comprising a cylinder outlet;
an electrochemical oxygen regulator comprising a regulator inlet and a regulator outlet, wherein the regulator inlet is connected to the cylinder outlet; and
a cannula connected to the regulator outlet.

17. The device of claim 16, further comprising:
a flow sensor.

18. The device of claim 16, further comprising:
a temperature sensor.

19. The device of claim 16, further comprising:
a pressure sensor.

20. The device of claim 16, further comprising:
a bandage, wherein the bandage comprises a diffuser.
